(19) United States
(12) Patent Application Publication
(10) Pub. No.: US 2007/0161551 A1
(43) Pub. Date: Jul. 12, 2007

(54) Methods and compositions for the treatment of lipodystrophy

(76) Inventor: Giampiero De Luca, Conches (CH)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/586,107

(22) PCT Filed: Jan. 26, 2005
(86) PCT No.: PCT/EP05/00758

§ 371 Date: Jul. 14, 2006

Related U.S. Application Data
(60) Provisional application No. 60/549,204, filed on Mar. 1, 2004.
(60) Provisional application No. 60/543,366, filed on Feb. 10, 2004.

(51) Int. Cl. A61K 38/27; A61K 31/401; A61K 31/366; A61K 31/22

(52) U.S. Cl. 514/12; 514/423; 514/460; 514/548

(57) ABSTRACT

The present invention is directed to methods and compositions for the treatment of lipodystrophy. The methods contemplate treatment of lipodystrophy in both HIV and non-HIV patients. More specifically, the methods are directed to a combination therapy that employs growth hormone and statins to effect treatment of lipodystrophy.

### BACKGROUND

1. Field of the Invention

The present invention is generally directed to methods and compositions for the treatment of lipodystrophy. More particularly, the instant invention is directed to combination therapy using compositions comprising growth hormone and one or more statin drugs in order to treat lipodystrophy.

2. Background of the Related Art

Lipodystrophy is a syndrome caused by a deficiency and/or destruction of adipocytes. The disorder is characterized by a selective loss of fat and is associated with hypertriglyceridemia, hepatic steatosis, and severe insulin resistance that often results in diabetes (Rossini et al., Metabolism., 26:637-650, 1977; Reitman et al., Trends Endocrinol. Metab., 11:410-416, 2000; Arioglu et al., Ann. Intern. Med. 133:263-274, 2000).

Congenital generalized lipodystrophy is an autosomal recessive disorder that is characterized by a deficiency of adipose tissue and accompanied by a severe resistance to insulin, leading to hyperinsulinemia, hyperglycemia, and an enlarged fatty liver (Seip et al., Acta Paediatr. Suppl. 413:2-28, 1996). In addition to this congenital disorder, lipodystrophy also may be acquired. Most significantly, it has been recognized that the dramatic clinical benefits of highly active antiretroviral therapy (HAART) are hindered by the development of HIV-lipodystrophy syndrome. This syndrome has received various designations, including HIV-associated dysmorphia/dysmetabolic syndrome (HADDS), the term by which it is referred to herein below. HIV-associated adipose redistribution syndrome (HARS) is considered as a subset of HADDS that is chiefly characterized by visceral lipohypertrophy.

HADDS is characterized by abnormal fat deposition, atrophy, metabolic complications, such as, hyperlipidemia, premature atherosclerotic lesions, and diabetes mellitus (Carr A et al., J Acquir Defic Syndr., 33:571-576, 2003), there may also be an associated depletion of lean body mass. This syndrome is now commonly encountered in over 60% of patients treated for HIV infection, particularly in those individuals being treated with HIV therapeutic agents, such as protease inhibitors as well as use of nucleoside reverse transcriptase inhibitors, such as stavudine. As indicated by Kravcik (HIV Clin. Trials, 1(3):37-50, 2000), to date, the pathogenesis of HIV-lipodystrophy syndrome remains largely unexplained and most therapies directed at fat changes have remained unsuccessful.

Thus, while HAART has effectively prolonged the lives of individuals with Acquired Immuno Deficiency Syndrome (AIDS), converting the disease to a chronic, high morbidity acquired HIV-related lipodystrophy syndrome, the incidences of this acquired form of lipodystrophy have increased alarmingly. (Carr et al., AIDS. 12:F51-F58, 1998; Carr et al., N Engl J Med. 339:1296, 1998, Carr et al., Lancet, 353:2093-2099, 1999, Miller et al., Lancet, 351:871-875, 1998; Vigouroux et al., Diabete Metab. 25:225-232, 1999). The abnormalities in lipid metabolism seen in this disorder also may lead to the increased incidence of accelerated atherosclerosis in HIV patients (Barbaro et al, Clin. Therap. 25(9):2405-18, 2003; Sklar et al., N Engl J Med., 349(21):2065-7, 2003; Friis-Moller et al., N Engl J Med., 349(21):1993-2003, 2003). Thus, HIV-related lipodystrophy is a multifactorial syndrome, and currently, there is no widespread treatment for this disease.

Atherogenic dyslipidemia (AD) is a central defect of the lipodystrophy syndrome, and also is recognized as an independent coronary heart disease risk factor. It is also associated with, and contributes to the pathogenesis of any of the conditions included in lipodystrophy syndrome, including, but not limited to hypertriglyceridemia (HTG), insulin resistance (IR), irnpaired glucose tolerance (IGT) and type 2 diabetes mellitus (DM-2). Because of the central role of AD in the progression of the metabolic abnormalities associated with HIV-related lipodystrophy, an effective treatment of AD is of utmost importance. Data for the HIV-uninfected population suggest lipodystrophy may further include high levels of total cholesterol, low-density-lipoprotein (LDL) cholesterol, and triglycerides, and low levels of high-density-lipoprotein (HDL) cholesterol. Because of these established risks, the National Cholesterol Education Program (NCEP) has issued guidelines for the treatment of AD for (non-HIV-infected) patients at risk.

In non-HIV patients, obesity is recognized as being associated with reduced growth hormone secretion (Veldhuis et al., J Clin Endocrinol Metab. 80:3209-3222, 1995; Veldhuis et al., J Clin Endocrinol Metab. 72:51-59, 1991; Ghigo et al., Metabolism. 41:560-563, 1992), where growth hormone concentrations have been shown to vary inversely with excess weight and body fat. HIV-lipodystrophy is different from normal obesity, because as discussed above, the fat deposition in HIV-lipodystrophy is redistributed and the lipodystrophic individuals do not tend to be overweight. Recently it was demonstrated that individuals with HIV lipodystrophy and increased accumulation of visceral fat also have a decreased growth hormone secretion (Rietschel et al., JCEM 86:504-510, 2001).

Thus, introduction of HAART with protease inhibitors and nucleoside reverse transcriptase inhibitors has greatly improved the life-expectancy of AIDS patients. Unfortunately, along with the increased life-expectancy, these patients increasingly develop secondary complications that lead to abnormal lipid distribution disorders. There is no doubt about the effectiveness of HAART, and it will, therefore, continue to be used to prolong the lives of AIDS patients. This, therefore leads to a need for providing additional therapies to manage the side-effects of HAART for the long long-term maintenance therapy of AIDS patients. Significant among these side-effects of the HAART that require amelioration are lipodystrophy and other HADDS-related metabolic dysfunctions. Lipodystrophy also is seen in non-HIV patients. Thus, there is a need to identify new and effective methods for the therapeutic intervention of both HIV-related and non-HIV related lipodystrophy.

### SUMMARY OF THE INVENTION

The present invention is directed to methods and compositions for the treatment of lipodystrophy. In specific embodiments, the present invention provides a method of treating a human suffering from an abnormal lipid distribution disorder, the method comprising administering to the subject a growth hormone and a statin-based therapeutic agent. Preferably, the statin-based agent and the growth hormone are provided in a single pharmaceutical composition. Other embodiments contemplate providing the statin-based agent in a first pharmaceutical composition and the growth hormone in a second pharmaceutical composition. Preferably, the growth hormone is recombinant growth hormone. However, the growth hormone may be one which has been isolated from an animal. The statin-based drug may be any statin-based agent known to those of skill in the art or any analog of a statin. Preferably, the statin-based agent is a lovastatin or a lovastatin analog. In exemplary embodiments, the statin-based drug is selected from the group consisting of atorvastatin, pravastatin, simvastatin, lovastatin, and fluvastatin.

In specific embodiments, the abnormal lipid distribution disorder is non-HIV-related lipodystrophy. In particularly preferred embodiments, the methods of the invention may be used to treat an HIV-related abnormal lipid distribution disorder. More particularly, the HIV-related abnormal lipid distribution disorder is selected from the atherogenic dyslipidemia, hypertriglyceridemia, elevated levels of cholesterol, elevated levels of low-density-lipoprotein cholesterol, and low levels of high-density lipoprotein cholesterol. In otlier embodiments, the method is used in the treatment of a subject who manifests a symptom associated with diabetes related adiposity. More particularly, the symptom of diabetes related adiposity is selected from the group consisting of insulin resistance, beta-cell dysfunction, loss of first phase insulin secretion, impaired glucose tolerance (IGT), elevated endogenous glucose production, excessive gluconeogenesis. In specific embodiments, the methods of the invention are used in the treatment of a subject is suffering from Type 2 Diabetes. The methods of the invention may advantageously involve treating the individual with a therapy traditionally used for the treatment of diabetes. For example, the method comprises administering an insulin secretagogue. Secretagogues used as anti-diabetic agents are well known to those of skill in the art and include, but are not limited to, sulphonylureas; tolbutamide; chlorpropamide; glimepiride; glipizide; glyburide; a meglitinides; repaglinide; pramlintide; morphilinoguanide; acetylcholine; a muscarinic agonist; carbachol; bethanechol; beta-L-glucose pentaacetate; chiro-inositol; myo-inositol; GIP; GLP-1; and Extendin-4. Derivatives, analogs and other molecules created by rational drug design based on these molecules may be used as the secretagogues.

In specific embodiments, the insulin secretagogue is a non-glucose dependent insulin secretagogue, and the combined effect of administering the growth hormone, statin and insulin secretagogue produces insulin release patterns capable of attaining glucose dependent, biphasic release characteristics with reduced likelihood of producing hypoglycemia.

In certain examples, the methods of the present invention may further comprise treating the subject with leptin.

Another aspect of the invention provides a therapeutic agent for use in combination therapy for an abnormal lipid distribution disorder, the composition comprising a first composition comprising a recombinant growth hormone in a pharmaceutically acceptable carrier, excipient or diluent; and a second composition comprising a statin-based drug in a pharmaceutically acceptable carrier, excipient or diluent. It is contemplated that the growth hormone and the statin-basec drug may be formulated in a single formulation. Preferably, however, the growth hormone is formulated in a separate formulation from the statin-based drug formulation. In specific embodiments, the growth hormone formulation and the statin-based drug formulation may be formulated as injectable formulations. In preferred embodiments however, the statin-based drug formulation is formulated for oral administration. In specific embodiments, the therapeutic agent may be formulated into a kit which contains the suitable implements for the administration of the various therapeutic components. The kit specifically may comprise lovastatin or an analog thereof as the statin-based drug. In specific embodiments, the statin-based drug of the therapeutic composition may be selected from the group consisting of atorvastatin, pravastatin, simvastatin, lovastatin, and fluvastatin. The compositions and methods of the invention may employ a single statin agent or alternatively may employ two or more such agents.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### DESCRIPTION OF THE PREFERRED EMBODIMENTS

Acquired lipodystrophy (or lipohypertrophy) is a significant problem in AIDS patients, leading to poor prognosis in individuals that are undergoing HAART to combat HIV infection. Other forms of lipodystrophy, e.g., obesity-related lipodystrophy that has attendant diabetes-related complications also provides a significant challenge to the current medical community. All of these lipodystrophies and disorders of lipid distribution in HIV and non-HIV patients may generically be referred to as “abnormal lipid distribution disorders.” The present invention provides new methods for the treatment of obesity. These therapeutic methods are based on the novel combination of growth hormone and statin-based drugs hyperlipidemic drugs such as Lipitor™ (atorvastatin), Pravachol™ (pravastatin), Zocor™ (simvastatin), Mevacor™ (lovastatin), and Lescol™ (fluvastatin. Both of these classes of drugs (i.e., growth hormone and statins) are well known to those of skill in the art and therefore the current recognition that these agents can be combined to achieve a therapeutically beneficial amelioration of the symptoms of lipodystrophy. This novel finding is exploited in the present invention to teach new methods and composition combinations for achieving such a therapeutic outcome. The disorders, methods and compositions are discussed in further detail herein below.

A. Disorders to be Treated by the Invention

As discussed herein above, the majority of HIV patients that are receiving HAART experience HADDS, which involves the pathological accumulation of adipose tissue in specific regional depots. The pathologic adipose tissue accumulation of HADDS may also be associated with abnormal adipose tissue depletion elsewhere (lipodystrophy or lipoatrophy), with or without associated metabolic abnormalities, premature atherosclerotic lesions, depletion of lean body mass, and/or other abnormal physiology. The methods of the present invention, which comprise administering a combination of growth hormone and statin drugs, results in the treatment of HADDS. This treatment is evidenced by a decrease, amelioration, or correction of any of the symptoms associated with HADDS.

Thus, in certain embodiments, it is contemplated that the methods of the invention will reduce the abnormal accumulation of adipose tissue in the abdomen, specifically in the visceral adipose tissue compartment (Miller et al, Lancet 21, 351(9106):871-875, 1998; Kotler et al., J Acquired Immun Defic Syndr 20:228-237, 1999; Kotler et al., HIV/AIDS 1999 Annual Update, 85-92, hiv.medscape.com, 1999; Engelson et al., Antiviral Therapy 4:(Sup 2):11 [Abstract 006], 1999; Englemson et al., Am J Clin Nutr 69(6):1162-1169, 1999) in patients that have this symptom. HADDS patients may also present with abnormal adipose tissue accumulation in the dorsocervical area (“buffalo hump”), the submandibular area (“horse collar”), the pectoral, mammary, and/or supraclavicular areas, and/or with subcutaneous lipomas (encapsulated benign fatty tumors, single or multiple). It is contemplated that the combination therapy of the invention which comprises at least a growth hormone composition and one statin-based therapeutic agent (e.g., Lipitor™, Pravachol™, Zocor™, Mevacor™, and Lescol™, or other analog of lovastatin) will decrease, or eliminate the abnormal adipose tissue so that there is a reduction in the size or amount of fatty deposit at one or more of the areas selected from the group consisting of abdominal (visceral) fat deposits, deposits in the dorsocervical area, submandibular area, the pectoral, mammary, supraclavicular areas, and/or in the subcutaneous lipomas.

In addition to ameliorating the above abnormal adiposity, the methods of the present invention also are directed to decreasing lipoatrophy in HADDS patients. HADDS patients are known to develop abnormally depleted subcutaneous adipose tissue, termed “peripheral lipodystrophy” (or lipoatrophy) at other specific sites. This adipose depletion is typically observed in the face (buccal, parotid, and periauricular fat pads), and in the subcutaneous adipose tissue surrounding the limbs, trunk, and/or gluteal regions. Thus, the present invention specifically contemplates methods of decreasing HADDS-associated subcutaneous lipid depletion by administering a combined therapy of growth hormone and at least one statin drug.

Some HADDS patients also present with metabolic abnormalities (Carr et al., AIDS 12:F51-58, 1998; Carr et al., Lancet 131:1881-1883, 1998; Carr et al., Lancet 353(9170):2093-2099, 1999; Carr et al., Antiviral Therapy 4(Sup 2):19 [Abstract 11], 1999; Lipodystrophy Rapid Report, 1999) associated with disordered lipid and/or glucose metabolism. Clinical manifestations may include fasting hypertriglyceridemia, hyperlipidemia, and abnormalities of the insulin/glucose axis (elevated fasting insulin, elevated C-peptide, insulin resistance or reduced insulin sensitivity), with or without overt diabetes (Carr et al., AIDS 12:F51-58, 1998; Carr et al., Lancet 131:1881-1883, 1998; Carr et al., Lancet 353(9170):2093-2099, 1999; Carr et al., Antiviral Therapy 4(Sup 2):19 [Abstract 11], 1999; Henry et al., Lancet 351:1328, 1998; Henry et al., Lancet 352:1031-1032, 1998; Grunfeld, Antiviral Therapy 4 (Sup 2):7 [Abstract 004], 1999). The methods of the invention are useful in treating one or more of these metabolic dysfunctions.

Another disorder that is specifically contemplated to be treated by the present invention include HADDS-related coronary heart disease (CHD). There are preliminary reports suggesting that patients with HADDS exhibit preclinical evidence of increased risk for coronary heart disease (CHD). Preclinical indicators of CHD include increased coronary artery calcification (CAC) as quantified by electron beam computed tomography (EBCT), and extracoronary indicators such as increased intima media thickening (IMT) in the carotid artery and impaired blood flow-mediated dilation in the brachial artery, as quantified by ultrasonography, which signify endothelial dysfunction that may lead to atherosclerosis and CHD. In eight patients with HADDS who developed increased abnormal girth with abnormally accumulated visceral adipose tissue after initiation of HIV protease inhibitor (PI) therapy who underwent EBCT, Kosmiski et al., Antiviral Therapy 4(Sup 2):49 [Abstract 056], (1999) reported a mean CAC score consistent with minimal identifiable plaque burden. There are also preliminary reports indicating that HIV patients receiving PIs display abnormal carotid IMT (Maggi et al., Antiviral Therapy 4(Sup 2):39 [Abstract 038], 1999) and impaired brachial flow-mediated dilation (Stein, Conference News Reports, AIDS Weekly via NewsRx.com (Nov. 22, 1999), signifying endothelial dysfunction. The therapeutic methods of the present invention may be used to treat one or more of the above-discussed symptoms of HADDS-related CHD.

Some patients with HADDS also exhibit involuntary weight loss with depletion of lean body mass (AIDS wasting or cachexia), and possibly depletion of lean body mass without overt weight loss (occult wasting). The methods of the invention may produce a useful weight gain in such individuals. Other abnormal physiology that may be treated in patients with HADDS or lipodystrophy syndrome using the methods of the present invention include gout and pancreatitis (presumably resulting from severe hypertriglyceridemia), hepatic steatosis (possibly reflecting chronic lactic acidosis), hypogonadism, and possibly other hormonal abnormalities (Henry et al., Lancet 351:1328, 1998; Henry et al., Lancet 352:1031-1032, 1998; Brinkman, Antiviral Therapy 4:(Sup 2):15 [Abstract 009], 1999; Lipodystrophy Rapid Report, 1999).

HADDS and lipodystrophy syndrome may or may not be associated with other abnormalities, such as cutaneous abnormalities (such as thinning hair, hair loss, hair brittleness, dry skin, abnormal nails, ingrown toenails), disorders of the coagulation syndrome that result in increased bleeding in hemophiliacs, osteoporosis or avascular necrosis of the hips, peripheral neuropathy, nausea, fatigue, weight loss, chronic diarrhea, fever, mennorhagia and menstrual abnormalities, impaired sexual dysfunction (decreased libido, erectile dysfunction), and abnormalities of the genitalia resembling Peyronie's Disease (Carr et al., AIDS 12:F51-58, 1998; Carr et al., Lancet 131:1881-1883, 1998; Carr et al., Lancet 353(9170):2093-2099, 1999; Carr et al., Antiviral Therapy 4(Sup 2):19 [Abstract 11], 1999; Lipodystrophy Rapid Report, 1999). The combined therapeutic regimen of the present invention may prove useful in ameliorating some or all of these HADDS-related pathologies.

In addition to lipodystrophy that manifests in HIV patients, the methods of the present invention also may be used to treat obesity-relate lipodystrophy in non-HIV subjects.

B. Compositions for Use in the Methods of the Invention

The methods of the present invention employ a combination of growth hormone and statin-related drugs in order to effect treatment of lipodystrophy. It is contemplated that any one or more of the symptoms of lipodystrophy exemplified herein above may be ameliorated by the use of this combination therapy. As both growth hormone and statin-related agents have previously been used in the treatment of other disorders, those of skill in the art will readily be able to adapt existing compositions and regimens for use in the present invention. Simply by way of example, the following section describes exemplary growth hormone and statin-related compositions that may be used in the present invention.

a. Growth Hormone

One of the active agents used in the therapeutic methods of the present invention is growth hormone. Preferably, the growth hormone used is human growth hormone. Human growth hormone, also known as somatotropin, is a protein hormone produced and secreted by the somatotropin cells of the anterior pituitary. Secretion is regulated by a releasing factor, i.e., the growth hormone-releasing hormone (GHRH), and by an inhibitory factor, somatostatin. Human growth hormone plays a key role in somatic growth through its effects on the metabolism of proteins, carbohydrates and lipids.

Human growth hormone is a single polypeptide chain of 191 amino acids (Bewley et al., Int J Pept Protein Res 4(4):281-287, 1972) having two disulfide bonds, one between Cys-53 and Cys-165, forming a large loop in the molecule, and the other between Cys-182 and Cys-189, forming a small loop near the C-terminus. The DNA sequence that confirmed the amino acid sequence was reported by Martial et al., Science 10;205(4406):602-607, 1979. Purified hGH is a white amorphous powder in its lyophilized form. It is readily soluble (concentrations >10 mg/L) in dilute aqueous buffers at pH greater than 7.2.

In solution, hGH exists predominantly as a monomer, with a small fraction as dimers and higher molecular weight oligomers. Under certain conditions, hGH can be induced to form larger amounts of dimers, trimers and higher oligomers. Several derivatives of hGH are known, including naturally-occurring derivatives, variants and metabolic products, degradation products primarily of biosynthetic hGH and engineered derivatives of hGH produced by genetic methods. One example of a naturally-occurring derivative of hGH is GH-V, a variant of growth hormone found in the placenta. Other members of the gene locus are described in Chen et al., Genomics 4(4):479-497, 1989.

Any derivative of hGH, including derivatives designed to be long-lasting in the body, can be used for the purpose of the present invention as long as it retains the biological activity of hGH. Further it is contemplated that mixed populations of GH derivatives also may be used.

Methionyl hGH, which was the first form of hGH to be produced through recombinant DNA technology will be useful in the present invention. This compound is a derivative of hGH having one additional methionine residue at its N-terminus (Goeddel et al., Nature 281(5732):544-548, 1979). Another GH that would be useful is naturally-occurring variant of hGH called 20-k-hGH. This variant of hGH has been reported to occur in the pituitary as well as in the bloodstream (Lewis et al., J Biol Chem 25;253(8):2679-2687, 1978; Lewis et al., Biochem Biophys Res Comm 29;92(2):511-516, 1980). This compound, which lacks the 15 amino acid residues from Glu-32 to Gln-46, arises from an alternative splicing of the messenger ribonucleic acid (DeNoto et al., Nucleic Acids Res 9(15):3719-3730, 1981). This compound shares many, but not all of the biological properties of hGH. This derivative may be used in the methods and combination therapy compositions of the present invention.

20-k-hGH is made in the pituitary and secreted into the blood. It makes up about 5% of growth hormone output of adults, and about 20% of growth hormone output of children. It has the same growth promoting activity as 22 kD growth hormone, and has been reported to have equal to, or greater than, the amount of lypolytic activity as the 22 kD form. It binds to growth hormone receptors with equal affinity as the 22 kD growth hormone, and has one tenth the lactogenic (prolactin-like) bioactivity as the 22 kD hormone. Unlike 22 kD, the 20-k-hGH has weak anti-insulin activity.

A number of derivatives of hGH arise from proteolytic modifications of the molecule. The primary pathway for the metabolism of hGH involves proteolysis. The region of hGH around residues 130-150 is extremely susceptible to proteolysis, and several derivatives of hGH having nicks or deletions in this region have been described (Thorlacius-Ussing, Neuroendocrinology, 45(3):233-242, (1987)). This region is in the large loop of hGH, and cleavage of a peptide bond in this region results in the generation of two chains that are connected through the disulfide bond at Cys-53 and Cys-165. Many of these two-chain forms are reported to have increased biological activity (Singh et al., Endocrinology 94(3):883-891, 1974). Many derivatives of human growth hormone have been generated artificially through the use of enzymes. The enzymes trypsin and subtilisin, as well as others, have been used to modify hGH at various points throughout the molecule (Lewis et al., Endocrinology 101(5):1587-1603, 1977; Graff et al., J Biol Chem 257:2365, 1982). One such derivative, called two-chain anabolic protein (2-CAP), was formed through the controlled proteolysis of hGH using trypsin (Becker et al, Abstract No. 342, 71^{st} Annual Meeting, The Endocrine Society, Seattle, Wash., June 1989). 2-CAP was found to have biological properties very distinct from those of the intact hGH molecule, in that the growth-promoting activity of hGH was largely retained and most of the effects on carbohydrate metabolism were abolished.

Asparagine and glutamine residues in proteins are susceptible to deamidation reactions under appropriate conditions. Pituitary hGH has been shown to undergo this type of reaction, resulting in conversion of Asn-152 to aspartic acid and also, to a lesser extent, conversion of Gln-137 to glutamic acid (Lewis et al., J Biol Chem 25;256(22):11645-11650, 1981). Deamidated hGH has been shown to have an altered susceptibility to proteolysis with the enzyme subtilisin, suggesting that deamidation may have physiological significance in directing proteolytic cleavage of hGH. Biosynthetic hGH is known to degrade under certain storage conditions, resulting in deamidation at a different asparagine residue (Asn-149). This is the primary site of deamidation, but deamidation at Asn-152 has been observed (Becker et al., Biotechnol Appl Biochem 10(4):326-337, 1988). Deamidation at Gln-137 has not been reported in biosynthetic hGH.

Methionine residues in proteins are susceptible to oxidation, primarily to the sulfoxide. Both pituitary-derived and biosynthetic hGH undergo sulfoxidations at Met-14 and Met-125 (Becker et al., Biotechnol Appl Biochem 10(4):326-337, 1988). Oxidation at Met-170 has also been reported in pituitary but not biosynthetic hGH. Both desamide hGH and Met14 sulfoxide hGH have been found to exhibit full biological activity (Becker et al., Biotechnol Appl Biochem 10(4):326-337, 1988). Truncated forms of hGH have been produced, either through the actions of enzymes or by genetic methods. 2-CAP, generated by the controlled actions of trypsin, has the first eight residues at the N-terminus of hGH removed. Other truncated versions of hGH have been produced by modifying the gene prior to expression in a suitable host. The first 13 residues have been removed to yield a derivative having distinctive biological properties (Gertler et al., Endocrinology 72118(2):720-6, 1986) in which the polypeptide chain is not cleaved.

The term “human growth hormone”, as used in the present invention, is intended to include the naturally-occurring derivatives, as noted above, including, without limitation, both the 20 kD and the 22 kD human growth hormone, GH-V, and other members of the growth hormone gene locus as described in Chen et al., Genomics 4(4):479-497, 1989. The term also includes functional derivatives, fragments, variants, analogs, or salts which retain the biological activity of growth hormone, i.e., which act as agonists to the growth hormone receptor. In other words, they are capable of binding to the growth hormone receptor to initiate the signaling activity of the receptor.

“Functional derivatives” as used herein covers derivatives which may be prepared from the functional groups which occur as side chains on the residues or the N- or C-terminal groups, by means known in the art, and are included in the invention as long as they remain pharmaceutically acceptable, i.e., they do not destroy the biological activity of hGH as described herein, i.e., the ability to bind the hGH receptor and initiate receptor signaling, and do not confer toxic properties on compositions containing the derivative. Derivatives may have chemical moieties, such as carbohydrate or phosphate residues, provided such a derivative retains the biological activity of hGH and remains pharmaceutically acceptable.

For example, derivatives may include aliphatic esters of the carboxyl groups, amides of the carboxyl groups by reaction with ammonia or with primary or secondary amines, N-acyl derivatives or free amino groups of the amino acid residues formed with acyl moieties (e.g., alkanoyl or carbocyclic aroyl groups) or O-acyl derivatives of free hydroxyl group (e.g., that of seryl or threonyl residues) formed with acyl moieties. Such derivatives may also include, for example, polyethylene glycol side-chains which may mask antigenic sites and extend the residence of the molecule in body fluids.

Of particular importance is a growth hormone that has been derivatized or combined with a complexing agent to be long lasting. For example, pegylated versions, or growth hormones genetically engineered to exhibit long lasting activity in the body, can be used to treat HADDS, or other abnormal lipid distribution disorder according to the present invention. Exemplary of hGH compositions with increased half-life include e.g., Nutropin Depot™ a slow releasing polyactide-coglycolide encapsulated hGH marketed by Alkermes/Genentech (Cook et al., J Clin Endocrinol Metab. 87(10):4508-14, 2002) and Albutropin™ (HGS), an albumin fusion of hGH that is in clinical trials (Osborn et al., Eur J Pharmacol.;456(1-3):149-58, 2002).

hGH that is acetylated at the N-terminus has been isolated and identified (Lewis et al., Endocrinology 104(5):1256-1265, 1979). It is not clear if acylation serves a regulatory role or is simply an artifact of the purification. However, it is expected that this the molecule exhibits anti-HADDS activity in a similar fashion to other hGH derivatives.

The term “salts” herein refers to both salts of carboxyl groups and to acid addition salts of amino groups of the hGH molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of hGH relevant to the present invention, i.e., the ability to bind to the hGH receptor and initiate receptor signaling.

A “fragment” of the growth hormone according to the present invention refers to any subset of the molecule, that is, a shorter peptide which retains the desired biological activity. Fragments may readily be prepared by removing amino acids from either end of the hGH molecule and testing the resultant for its properties as an hGH receptor agonist. Proteases for removing one amino acid at a time from either the N-terminal or the C-terminal of a polypeptide are known, and so determining fragments which retain the desired biological activity involves only routine experimentation.

Additionally, the polypeptide which has such hGH receptor agonist activity, be it hGH, an analog or variant, salt, functional derivative or fragment thereof, can also contain additional amino acid residues flanking the hGH polypeptide. As long as the resultant molecule retains the hGH receptor agonist ability of the core polypeptide, one can determine whether any such flanking residues affect the basic and novel characteristics of the core peptide, i.e., its receptor agonist characteristics, by routine experimentation. The term “consisting essentially of”, when referring to a specified sequence, means that additional flanking residues can be present which do not affect the basic and novel characteristic of the specified sequence.

A “variant” of the human growth hormone according to the present invention refers to a molecule which is substantially similar to either the entire peptide or a fragment thereof. Variant peptides may be conveniently prepared by direct chemical synthesis of the variant peptide, using methods well known in the art. Of course, a variant human growth hormone would preferably have similar hGH receptor binding and signal initiating activity as hGH and which would, therefore, be expected to have similar anti-HADDS activity to hGH.

Amino acid sequence variants of the human growth hormone can be prepared by mutations in the DNAs which encode the synthesized human growth hormone derivatives. Such variants include, for example, deletions from, or insertions or substitutions of, residues within the amino acid sequence. Any combination of deletion, insertion, and substitution may also be made to arrive at the final construct, provided that the final construct possesses the desired activity. Obviously, the mutations that will be made in the DNA encoding the variant peptide must not alter the reading frame and preferably will not create complementary regions that could produce secondary mRNA structure (see European Patent Publication No. EP 75,444, the entire contents of which are hereby incorporated by reference).

At the genetic level, these variants ordinarily are prepared by site-directed mutagenesis (as exemplified by Adelman et al., DNA 2(3):183-193, 1983) of nucleotides in the DNA encoding the peptide molecule, thereby producing DNA encoding the variant, and thereafter expressing the DNA in recombinant cell culture. The variants typically exhibit the same qualitative biological activity as the non-variant peptide.

An “analog” of human growth hormone according to the present invention refers to a non-natural molecule which is substantially similar to either the entire molecule or to an active fragment thereof. An analog of human growth hormone useful in the present invention would preferably exhibit anti-HADDS activity, at least when administered in combination with a statin, but preferably when administered alone.

Examples of production of amino acid substitutions in proteins which can be used for obtaining analogs of the hGH for use in the present invention include any known method steps, such as presented in U.S. Patents RE 33,653; U.S. Pat. Nos. 4,959,314; 4,588,585 and 4,737,462, to Mark et al.; U.S. Pat. No. 5,116,943 to Koths et al.; U.S. Pat. No. 4,965,195 to Namen et al.; and U.S. Pat. No. 5,017,691 to Lee et al., and lysine substituted proteins presented in U.S. Pat. No. 4,904,584 (Shaw et al.).

Among the substances which bind to, and initiate, signaling of the human growth hormone receptor which may be used in accordance with the present invention are all of those growth hormone analogs and mimetics already known in the literature, such as, for example, are disclosed in U.S. Pat. Nos. 5,851,992; 5,849,704; 5,849,700; 5,849,535; 5,843,453; 5,834,598; 5,688,666; 5,654,010; 5,635,604; 5,633,352; 5,597,709; and 5,534,617.

Preferably, the hGH variant or analog will have a core sequence, which is the same as that of the native sequence or biologically active fragment thereof, which has an amino acid sequence having at least 70% identity to the native amino acid sequence and retains the biological activity thereof. More preferably, such a sequence has at least 80% identity, at least 90% identity, or most preferably at least 95% identity to the native sequence.

Although human growth hormone was originally obtained from pituitary glands of cadavers, these preparations were not electorophoretically homogeneous, and antibodies appeared in the serum of patients treated with preparations of the order of 50% purity, the immunogenicity being attributed to inactive components. Recombinant DNA technology permitted production of an unlimited supply of hGH in a number of different systems. Purification of hGH from the culture medium is facilitated by the presence of only low amounts of contaminating proteins. In fact, it has been shown that hGH can be purified on a laboratory scale by a single purification step on a reversed-phase HPLC column (Hsiung et al., Biotechnology 7:267, 1989).

Recombinant human growth hormone, rhGH, is produced by Serono S. A., as SEROSTIM®, which product has been given FDA approval for treating weight loss and wasting in HIV patients. PROTROPIN®, produced by Genentech, Inc. (South San Francisco, Calif.), differs slightly in structure from natural sequence hGH, having an additional methionine residue at the N-terminus. Recombinant hGH is generally marketed as vials containing hGH plus additional excipients, e.g., glycine and mannitol, in a lyophilized form. A companion diluent vial is provided, allowing the patient to reconstitute the product to the desired concentration prior to administration of the dose. Recombinant hGH can also be marketed in other well-known manners, such as prefilled syringes, etc.

In specific embodiments, it is contemplated that that methods described herein will use SEROSTIM®, a recombinant human growth hormone (rhGH) produced by Serono S. A. This product has recently been given full FDA approval for treating wasting syndrome in patients with HIV-associated wasting or cachexia. Such growth hormone compositions will be useful in the methods of the present invention. Previously, it has been noted that many patients with overt AIDS wasting are able to tolerate relatively high doses (6 mg/day) of recombinant growth hormone (rhGH; Serono's SEROSTIM®) administered subcutaneously (s.c.), without developing adverse effects that require dose reduction or cessation of therapy (Schambelan et al., Ann Intern Med 125(11):873-882, 1996).

It is contemplated that those of skill in the art can vary the dose of rhGH and monitor the patient for development of adverse symptoms from the rhGH administration. It is specifically contemplated that the subject will receive, 1 mg/day, 2 mg/day, 3 mg/day, 4 mg/day, 5 mg/day, 6 mg/day, 7 mg/day, 8 mg/day, 9 mg/day, 10 or more rhGH on a daily basis. Such therapy may be administered in a single dose or it may alternatively be divided into multiple doses to be administered at set intervals during the day. It also is contemplated that the GH composition may be administered at time intervals other than daily. For example, the subject may be given the GH therapy every other day or every week. The symptoms to be monitored to assess the adverse effects of the GH include, but are not limited to, tissue turgor, joint stiffness, arthalgias, and/or paresthesias. The clinician will be able to use such symptoms as guidance parameters to assess whether a given dose of GH should be adjusted (up or down).

Currently, those of skill in the art are conducting dose-ranging trials to investigate the additional effective and safe doses of rhGH for patients with HADDS. Thus, doses of GH for use in AIDS patients are well known to those of skill in the art. Doses from such studies may readily be adapted for use in the methods described herein.

Windisch et al., Ann Pharmacother 32(4):437-445, 1998, reported that HIV-associated wasting was characterized by weight loss, depletion of lean body mass and preservation of body fat, leading to muscle weakness and organ failure. Although the FDA has approved recombinant growth hormone for treating HIV-associated wasting, the adverse event profile is similar to that of other recombinant growth hormone products. Trials of recombinant growth hormone on the control of wasting in patients with HIV/AIDS have been encouraging. Post-marketing experience with over 10,000 HIV/AIDS wasting patients receiving SEROSTIM® since 1996 reveals that a three-month course of therapy was effective in the majority of patients with AIDS wasting.

Given the previous findings seen with SEROSTIM®'s effects on AIDS wasting, it is contemplated that the therapeutic methods of the present invention, which contemplate a combined therapy in which rhGH is administered in combination with at least one statin drug, may be used periodically to control HIV-related, or other abnormal lipid distribution disorder. For example, such periodic therapy would entail treating the patient with a course of the combination therapy for a period of 1, 2, 3, 4, 5 or 6 months. Alternatively, in patients with HIV-related lipodystrophy or HADDS, the methods of the present invention may be used as a continuous therapy in conjunction with HAART to control the adverse side effects of HAART that manifest in lipodystrophy, HADDS, and the like. It should also be understood that, to be useful, the treatment provided need not be absolute, provided that it is sufficient to carry clinical value. An agent which provides treatment to a lesser degree than do competitive agents may still be of value if the other agents are ineffective for a particular individual, if it can be used in combination with other agents to enhance the overall level of protection, or if it is safer than competitive agents.

During the therapy it would be advantageous to monitor the symptoms of the patient to ensure that adverse effects from the GH are not being experienced. In the event that adverse effects are not seen and the combined therapy is not producing a fast enough therapeutic outcome, a more aggressive course of therapy may be administered wherein the dose of the rhGH and/or the statin drug is increased. In the event that the therapy produces side effects such as tissue turgor, joint stiffness, arthalgias, and/or paresthesias, the dose of the rhGH may be reduced to alleviate the side effects.

Krentz et al., J Acquir Immune Defic Syndr 6(3):245-251, 1993, compared metabolic and anthropometric changes induced by recombinant human growth hormone dosed at 5.0 mg versus 2.5 mg every other day (qod) in 10 patients with HIV/AIDS. During treatment, insulin-like growth factor-I (IGF-I) levels increased significantly in the pharmacological rhGH treatment group receiving 5.0 mg qod, whereas no significant change was observed in IGF-I in the group receiving 2.5 mg qod of rhGH. In the group treated with 5.0 mg qod dose of hGH, weight loss preceding the study was reversed in each of the four patients who completed the study. This weight gain was associated with increases in lean body mass and total body water, and with concomitant decreases in fat mass and urinary nitrogen excretion.

In a large, randomized, placebo-controlled study, Schambelan et al., Intern Med 125(11):873-882, 1996, used dual X-ray absorptiometery (DXA) scanning to evaluate changes in body composition produced by administration of recombinant human growth hormone dosed at 0.1 mg/kg/day (or 4 to 6 kg per day, depending on patient weight) compared to placebo over a 12 weeks course of therapy. By the end of treatment, significant increases in lean body mass and weight are observed in the rhGH group, compared to the placebo group, and these increases correlated with improvements in physical function (treadmill performance). The rhGH therapy was associated with minor increments in fasting plasma glucose, which were of negligible clinical significance.

The studies of Krentz et al., J Acquir Immune Defic Syndr 6(3):245-251, 1993 and Schambelan et al., Intern Med 125(11):873-882, 1996, may readily be repeated with the combination of GH/statin-based therapeutic methods of the present invention. Such determinations would be merely routine, and would show the beneficial effects of the reducing, ameliorating or otherwise improving one or more symptoms of lipodystrophy.

b. Statin Drugs

As discussed herein throughout, the therapeutic methods for the present invention employ a second compound in addition to the GH. The second active compound in the combination therapy of the present invention is a statin-related agent. By “statin-related” agent or drug the present application refers to any statin drug that is presently on the market, or is modified from the presently marketed statin drugs, and has a therapeutic effect when combined with the growth hormone compositions used in the invention. As such it should be understood that analogs and variants of preexisting statins are contemplated to be useful herein. Such analogs or variants may be produced through rational drug design techniques known to those of skill in the art. In particular, statin drugs are known as HMGCoA reductase inhibitors. These drugs are presently in clinical use as drugs in the battle against high cholesterol and in the control of heart attacks, both recurrent and first heart attacks. These agents generally have few side effects, and help not only to lower overall cholesterol, LDL cholesterol and triglycerides, but also to increase HDL cholesterol.

Statins are exemplified by lovastatin (CAS Registry No. 75330-75-5; also known as mevinolin or monacolin K), and analogs of this compound have been described in numerous publications and patents. Exemplary statin compositions that are commercially available include Lipitor™ (atorvastatin), Pravachol™ (pravastatin), Zocor™ (simvastatin), Mevacor™ (lovastatin), and Lescol™ (fluvastatin). Methods of preparing such compounds are well known to those of skill in the art (see e.g., U.S. Pat. Nos. 6,521,762; 4,420,491; 4,342,767; 4,319,039; 4,294,846; 4,444,784; 4,582,915 and 4,820,850). As described in the foregoing patents, statins are traditionally produced through fermentation using organisms from the Aspergillus genus, Monascus genus, Pleurotus genus, Coniothyrium genus and the like (see U.S. Pat. No. 6,521,762 for review of such fermentation procedures).

Moreover, formulations of statins as pharmaceutical medicament have been described in e.g., the Physician's Desk Reference. For example, tablet formulations of Lipitor™ (atorvastatin calcium) are described at pages 2547-2551 (Parke-Davis, NJ.) and 2610-2613 (Pfizer, N.Y.) of the Physician's Desk Reference(57^{th} Edition, 2003). These formulations are supplied as tablets of atorvastatin calcium containing 10 mg, 20 mg, 40 mg, 50 mg, and 80 mg atorvastatin. The tablets are administered in doses ranging from 10 mg/day to 80 mg/day. The compositions of Lipitor™ presently being used to lower cholesterol in humans may be used in the combined treatments of the present invention to produce a therapeutic amelioration of HADDS and related lipodystrophy.

Pravachol™ (pravastatin sodium; Bristol-Myers Squibb, NY), is another exemplary commercially available statin that may be used in the combined therapies of the present invention. Pravachol™ is supplied as a 10 mg, 20 mg, 40 mg, and 80 mg tablets. These tablets may be administered at a daily dose of ranging from 10 mg/day to 80 mg/day. In exemplary treatments for hypercholesterolemia, 40 mg/day are administered as a single daily dose, with or without food. However, it is generally appreciated that this dose may be increased or lowered depending on the level of renal and liver function of the patient being treated. The administration doses and treatment guidelines for Pravachol™ are discussed in further detail at pages 1101-1105 of the Physician's Desk Reference(57^{th} Edition, 2003) and may be used to provide guidance for the use of statins in the methods of the present invention.

Zocor™ (simvastatin; Merck & Co., Inc., NJ), is another exemplary statin composition that may be used in the present invention. Formulations of this statin are described at pages 2126-2131 of the Physician's Desk Reference(57^{th} Edition, 2003). The daily doses may range from 5 mg/day to 80 mg/day and those of skill in the art are referred to the Physician's Desk Reference for further guidance regarding treatment protocols that may be used and/or modified for the present invention. It is contemplated that doses and treatment protocols that are useful for lowering cholesterol will also be useful in the treatment of HADDS described in the present application.

Mevacor™ (lovastatin; Merck & Co., Inc. NY), and Lescol™ (fluvastatin) are other exemplary statins that are described in the Physician's Desk Reference(57^{th} Edition, 2003) at pages 2036-2041 and 2283-2287, respectively. Those of skill in the art will readily be able to modify the above-referenced pharmaceutical compositions that comprise various statin-related agents for the methods of the present invention.

For treatment protocols, those of skill may use the guidelines used for the any of the above-referenced pharmaceutical statins. Administration of ordinary tablets containing statin once, twice, three or more times a day. Accordingly, the skilled artisan may use dosages that have previously proven effective for the above indications as a preliminary measure of the amount of any of the above-referenced statins, to use in the therapeutic methods of the invention.

Oral doses of the statins are particularly contemplated. Such oral doses may comprise the administration of between about 5 mg to about 80 mg statin drug on a daily basis. However, larger doses e.g., up to 200 mg/day also may be used. Thus, the subject may receive 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg or more statin drug orally. Of course it should be understood the subject may receive more or less of the statin. Also it should be understood that similar doses may be administered through other routine routes of administration. The statin may be delivered in a single dose or alternatively may be subdivided and administered in multiple doses over a given period of time.

C. Combination Therapies with Additional Therapeutic Agents

The methods of the invention involve the combined use of growth hormone and statin-related compounds. However, in addition to therapies based solely on the delivery of GH/statin combination therapy, the methods of the present invention also contemplate combination therapy with a third composition that specifically targets one or more of the symptoms of lipodystrophy. In the context of the present invention, it is contemplated that GH/statin-based methods could be used similarly in conjunction with other agents for e.g., treating obesity, diabetes and the like. Such additional therapeutic compounds also may comprise compositions that enhance the effects of growth hormone and/or the statin-related agents.

In particular embodiments, it is contemplated that the growth hormone/statin treatment in accordance with the present invention may be supplemented with the administration of a substance which stimulates production of endogenous growth hormone either directly or indirectly by suppressing endogenous somatostatin secretion. It is known that human growth hormone releasing hormone (hGHRH) stimulates the release of hGH. Thus, the biological activity of hGH can be indirectly obtained by administering GHRH or a functional derivative, salt, variant, analog or fragment thereof which retains the biological activity of GHRH, i.e., the ability to stimulate the release of growth hormone. Thus, for example, besides GHRH there may be used functional derivatives thereof in accordance with the above definition, analogs or variants thereof, which have at least 70% sequence identity, more preferably 80% or 90% or, most preferably, 95% sequence identity therewith, yet retains the biological activity of GHRH, or a variant or analog which is a polypeptide encoded by a DNA which hybridizes to the native DNA encoding GHRH under moderately stringent conditions, or preferably under highly stringent conditions, all in accordance with the definitions given hereinabove. Any of the GHRH or GHRH analogs or agonists known in the literature and disclosed as simulating the release of growth hormone can be used in the present invention, such as those disclosed in U.S. Pat. Nos. 5,792,747; 5,776,901; 5,696,089; 5,137,872; 5,767,085; 5,612,470; 5,846,936; and 5,847,066. See also Thorner et al., Recent Prog Horm Res., (1997), Felix et al., Int J Pept Protein Res., 46(3-4):253-64 (1995), Alba-Roth et al J. Clin. Endo. Metab., 67, 1186-1189 (1988); Friend et al., Eur J Endocrinol., 137(4):377-86 (1997).

Other substances capable of promoting the release of growth hormone in vivo which can be used in accordance with the present invention include those disclosed in U.S. Pat. Nos. 5,807,985; 5,604,578; 5,795,957; 5,777,112; 5,767,118; 5,731,317; 5,726,319; 5,726,307; 5,721,251; 5,721,250, etc.

There can also be used in accordance with the present invention any other molecule which binds to receptors on pituitary somatotrophes and initiates signaling of that receptor. It is known, for example, that small molecules, sometimes called secretagogues, have been developed which bind GHRS receptors and cause them to initiate signaling, which signal initiation is the same as one obtains with natural ghrelin binding to the receptor. Such molecules are known, for example, from U.S. Pat. Nos. 5,773,441; 5,798,337; 5,630,433; 5,767,124; and 5,723,616. See also Bowers et al Endocrinology, 128:2027-2035 (1991), Thorner et al., Recent Prog Horm Res., 52:215-46 (1997), Camanni et al., Front Neuroendocrinol. 19(1):47-72, (1998), Ankersen et al., J Med Chem., 41(19):3699-704, (1998), Smith et al., Science, 260(5114):1640-3 (1993) and Ghigo et al., Horm Res., 51 Suppl 3:9-15 (1998). Thus, the present invention is intended to include any substance which binds to GHRS receptor and initiates signaling thereof so as to obtain the same ultimate qualitative effect as the administration of natural hGH, insofar as the treatment of HADDS is concerned.

In other embodiments, it is contemplated that the growth hormone/statin-based combination therapy of the invention is supplemented with a therapeutic regimen that treats obesity-related diabetes. Such a therapeutic regimen may comprise administering an insulin secretagogue to said subject. The use of such secretagogues for the amelioration of diabetes is well known to those of skill in the art. Classes of secretagogues that may be used include but are not limited to sulphonylurea; tolbutamide; chlorpropamide; glimepiride; glipizide; glyburide; a meglitinides (see Physician's Desk Reference 56^{th} Edition, page 2432 for description of an exemplary meglitinide pharmaceutical formulation); repaglinide; pramlintide; morphilinoguanide; acetylcholine; a muscarinic agonist; carbachol; bethanechol; beta-L-glucose pentaacetate; chiro-inositol; myo-inositol; GIP; GLP-1; and Extendin-4. Those of skill in the art are referred to Goodman & Gilman's The Pharmacological Basis of Therapeutics, Eds. Hardman et al., 9^{th} Edition, Chapter 60 which describes insulin and oral hypoglycemic agents that could be used in conjunction with the present invention. Particularly preferred oral hypoglycemics include sulphonylureas (described at pages 1507-1510 of Goodman & Gilman. See also see Physician's Desk reference, 56^{th} Edition pages 717, 741, 2680, 2692, 2693, and 1086 for descriptions of exemplary sulphonylurea pharmaceutical formulations currently being used). It is contemplated that the metformin (see Physician's Desk reference, 56^{th} Edition page 1080 for description of an exemplary metformin pharmaceutical formulation), phenformin or other biguanides also may be used. Thiazolidendiones, such as ciglitazone and pioglitazone, also may prove useful in the methods of the present invention. Those of skill in the art also are referred to the Physician's Desk Reference, 56^{th} Edition pages 3275 and 1490 for descriptions of exemplary thiazolidendione pharmaceutical formulations currently being used. Diazoxide, an antihypertensive agent also is known as a potent antilhyperglycemic agent. It is likely that, as certain statin compounds such as Liptor™ are HMG-CoA reductase inhibitors, additional such inhibitors will be identified in such additional inhibitors may be used in the present invention to produce an additional therapeutic effect against lipodystrophy in accordance with the present invention. Such additional agents may or may not be analogs of lovastatin, as long as they act as inhibitors of HMG-CoA reductase in a manner similar to lovastatin analogs.

Leptin (Zhang et al., Nature 372:425, 1994) is a protein hormone with important effects in regulating body weight, metabolism and reproductive function (see e.g., U.S. Pat. No. 5,935,810). The protein is approximately ˜16 kDa in mass and encoded by the obese (ob) gene. Leptin is expressed predominantly by adipocytes but leptin also is secreted by cells in the epithelium of the stomach and in the placenta. Leptin receptors are highly expressed in areas of the hypothalamus known to be important in regulating body weight, as well as in T lymphocytes and vascular endothelial cells. Studies with obese and non-obese humans have shown a strong positive correlation of serum leptin concentrations with percentage of body fat, and also that there was a higher concentration of ob mRNA in fat from obese compared to thin subjects. It appears that as adipocytes increase in size due to accumulation of triglyceride, they synthesize more and more leptin. Daily injections of recombinant mouse or human leptin into ob/ob mice (i.e., the obese mouse mutants that are unable to synthesize leptin) led to a dramatic reduction in food intake within a few days, and to roughly a 50% reduction in body weight within a month. Moreover, when leptin is given to normal mice, they lose weight, show profound depletion of adipose tissue and manifest increases in lean mass. It has been shown that treatment with leptin promotes lipolysis in adipose tissue, but has no apparent effect on lean tissue. Given these results, it is contemplated that the rhGH/statin-based therapies of the present invention may advantageously be supplemented with a therapeutic regimen that provides the subject with leptin.

To achieve the appropriate therapeutic outcome in the combination therapies contemplated herein, be it a decrease in girth of the area at which the abnormal fat deposition has taken place, an improvement in the appearance of the area of the body where the fat has become depleted, a reduction in hypoglycemia, an increase in insulin secretion or production, or other parameter, one would generally administer to the subject the GH and the statin-related composition and at least one other therapeutic agent (third therapeutic agent). These compositions would be provided in a combined amount effective to produce the desired therapeutic outcome. This process may involve administering the rGH therapy, the statin-based therapeutic composition and the third therapeutic composition at the same time. This may be achieved by administering a single composition or pharmacological formulation that includes all of the active agents, or by administering to the subject three distinct compositions or formulations, at the same time, wherein one composition includes the rhGH, the second compositions includes the statin-related agent, and the third composition includes the third therapeutic agent.

Alternatively, the rhGH treatment may precede or follow statin-based therapy and/or the third agent treatment by intervals ranging from minutes to weeks. In embodiments where two or more of the therapeutic compositions are administered separately, one would generally ensure that a significant period of time did not expire between the time of each delivery, such that the statin-based agent and rhGH and/or the third agent would still be able to exert an advantageously combined effect on the cell. In such instances, it is contemplated that one would administer all three compositions within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

D. Pharmaceutical Compositions

Pharmaceutical compositions for administration according to the present invention can comprise at least one formulation of human growth hormone according to the present invention in a pharmaceutically acceptable form optionally combined with a pharmaceutically acceptable carrier. These compositions can be administered by any means that achieve their intended purposes. Amounts and regimens for the administration of a composition according to the present invention can be determined readily by those with ordinary skill in the art for treating HADDS, or other abnormal lipid distribution disorder. As discussed above, those of skill in the art could initially employ amounts and regimens of GH currently being used in a medical context. To this effect, those skilled in the art are specifically referred to each of the entries in the Physician's Desk Reference, 56^{th} Edition, at pages 2818-2820 (GENOTROPIN®), 3215-3215 (GEREF®), 1930-1934 (HUMATROPE®), 2419-2421 (NORDITROPIN®), 1417-1425 (NUTROPIN®), 3225-3226 (SAIZEN®), and 3229-3231 (SEROSTIM®) each incorporated herein by reference. Each of these entries in the Physician's Desk Reference provide exemplary guidance as to types of formulations, routes of administration and treatment regimens that may be used in administering GH. Any of the protocols, formulations, routes of administration and the like described therein can readily be modified for use in the present invention.

Compositions within the scope of this invention include all compositions comprising at least one human growth hormone or derivative, analog, or variant thereof according to the present invention in an amount effective to achieve its intended purpose. Similarly, as the therapeutic methods of the present invention contemplate a combination therapy in which statin-based agents are administered in addition to the human growth hormone-based therapy, the pharmaceutical compositions of the invention also contemplate all compositions comprising at least one statin-based therapeutic agent, or analog thereof in an amount effective to achieve the amelioration of one or more of the symptoms of lipodystrophy when administered in combination with the human growth hormone.

While individual needs vary, determination of optimal ranges of effective amounts of each component is within the skill of the art. Typical dosages of the growth hormone comprise about 0.01 to about 0.1 mg/kg body weight per day, which will usually amount to about 1-6 mg/day, subcutaneously for e.g., 5 to 12 weeks. Of course, those of skill in the art may choose a treatment regimen that lasts longer, e.g., up to 48 weeks. When administered to AIDS patients, the hGH anti-HADDS therapy may be administered concomitantly with other AIDS therapies or other therapies designed to alleviate the symptoms of lipodystrophy as discussed herein above. Since supraphysiologic doses of hGH (>5 mg/day) have been safely administered to AIDS wasting patients continuously on a daily basis as s.c. injections for periods of two to four years, it is contemplated that the combined therapies which employ growth hormone and at least one statin agent also may be effective over such periods. While continuous, daily administration is contemplated, it may be desirable to ceases the combined therapy when the symptoms of lipodystrophy are alleviated. Of course, the therapy may be reinitiated in the event that abnormal adipose tissue re-accumulates.

It is understood that the suitable dose of a composition according to the present invention will depend upon the age, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight.

As discussed above, the total dose required for each treatment may be administered in multiple doses or in a single dose. The compositions may be administered alone or in conjunction with other therapeutics directed to the disease or directed to other symptoms thereof.

As is apparent from the disclosure presented herein, in a broad aspect the present application contemplates clinical application of a combination therapy comprising a first composition that contains a growth hormone formulation, and a second composition that contains a statin-based drug. Therefore, the compositions should be formulated into suitable pharmaceutical compositions, i.e., in a form appropriate for in vivo applications in such combination therapies. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of each of the therapeutic agents being used, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also can be incorporated into the compositions.

The active compositions of the present invention include classic pharmaceutical preparations of growth hormone which have been discussed herein as well as those known to those of skill in the art. Statins, such as, e.g., Lipitor™ and the like, also are known to those of skill in the art. Administration of these compositions according to the present invention will be via any common route so long as the target tissue is available via that route. Most commonly, these compositions are formulated for oral administration. However, other conventional routes of administration, e.g., by subcutaneous, intravenous, intradermal, intramusclar, intramammary, intraperitoneal, intrathecal, intraocular, retrobulbar, intrapulmonary (e.g., term release), aerosol, sublingual, nasal, anal, vaginal, or transdermal delivery, or by surgical implantation at a particular site also may be used particularly when oral administration is problematic. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the therapeutic agents of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. Examples of preferred doses of the growth hormone and the statin have been discussed above. Parenteral administration of one or both of the therapeutic compounds may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435 1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

In certain embodiments, the growth hormone or other protein may be administered using gene therapy embodiments that employ viral delivery, the unit dose may be calculated in terms of the dose of viral particles being administered. Viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10 to 100-fold) due to the presence of infection defective particles.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

E. Examples

The following example(s) is included to demonstrate preferred embodiments of the invention. It should be appreciated by those of skill in the art that the techniques disclosed in the example(s) that follows represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

AIDS patients with a history of long-term use (an average of 12 months) of HAART that manifest symptoms of HADDS, including buffalo humps, central adiposity and peripheral muscle wasting associated with fatigue, along with elevated levels of plasma triglycerides and/or cholesterol are selected for the study.

Therapy with rhGH (SEROSTIM®) is initiated in all patients at a dose of e.g., 4 mg/day subcutaneously (other concentrations, e.g., 6 mg/day could be used). At the same time, the patients are treated with a statin in a dosage amount of 40 mg/day, which is administered orally. This dosage amount is based on the average dose of statin drugs commonly administered for antilipemic purposes. The patients are maintained on this regimnen for three months and monitored every two weeks for improvements of fat maldistribution. In parallel therapies, patients are treated with just rhGH and just statin. After 3 months notable improvements in fat maldistribution, with 25-75% reduction in buffalo hump syndrome and abdominal girth, but no change in peripheral lipodystrophy is seen in the combined treatment with rhGH and statin. Individuals receiving rhGH or statin alone do not experience as dramatic an improvement in fat maldistribution as seen with the combined therapy. Weights were stable, and there were no consistent changes in total body fat and blood lipids, despite 5-10% gain in fat-free mass.

An effective treatment with the methods of the present invention is any notable reduction in the size of, and firmness of, the buffalo hump and truncal adiposity. Preferably, the reduction in size and firmness of buffalo hum and truncal adiposity that is seen with the combined rhGH/statin treatment is greater than that seen in patients with HADDS that are treated with rhGH alone (Torres et al., Abstract 32164: 12^{th} World AIDS Conference, Geneva [Abstract 32164], 1998; Torres, Abstract 675: 6^{th} Conference on Retroviruses and Opportunistic Infections [Abstract 675], 1999; Wanke et al., AIDS 13(15):2099-2103, 1999; Mauss et al., Antiviral Therapy 4(Sup 2):27 [Abstract 018], 1999; Engleson et al., Antiviral Therapy 4:(Sup 2):11 [Abstract 006], 1999; Engleson et al., Am J Clin Nutr 69(6):1162-1169, 1999; Milano et al., Antiviral Therapy 4(Sup 2):41 [Abstract 042], 1999). Torres, Abstract 675: 6^{th} Conference on Retroviruses and Opportunistic Infections [Abstract 675], 1999, has reported that, by four months, rhGH therapy dosed at 4 to 6 mg/day significantly reduces the size and firmness of buffalo humps, and reduces truncal adiposity, with no change in peripheral lipodystrophy, while fat free mass increased 5 to 10%. There were no significant or consistent changes in body weight, total body fat, or blood lipids during the treatment period.

Collectively, these clinical studies cited above demonstrate that therapy with rhGH (SEROSTIM®), administered subcutaneously, in doses ranging from 3 to 6 mg per day for 12 to 24 weeks significantly reduces abnormally accumulated fat, compared to baseline. Specifically, SEROSTIM® (rhGH) has been shown to reduce abdominal girth (Wanke et al., AIDS 13(15):2099-2103, 1999), visceral adiposity (Engleson et al., Antiviral Therapy 4:(Sup 2):11 [Abstract 006], 1999; Engleson et al., Am J Clin Nutr 69(6):1162-1169, 1999; Mauss et al., AIDS 12(Sup 4):145, 1998), buffalo hump (Torres, Abstract 32164: 12^{th} World AIDS Conference, Geneva [Abstract 32164], 1998; Torres et al., Abstract 675: 6^{th} Conference on Retroviruses and Opportunistic Infections [Abstract 675], 1999), and solitary lipomas (Milano et al., Antiviral Therapy 4(Sup 2):41 [Abstract 042], 1999). Therapy with rhGH (SEROSTIM®) also increased lean body mass and body cell mass as quantified by biolectrical impedance analysis (Wanke et al., AIDS 13(15):2099-2103, 1999; Engleson et al., Antiviral Therapy 4:(Sup 2):11 [Abstract 006], 1999; Engleson et al., Am J Clin Nutr 69(6):1162-1169, 1999). The use of the statin drug in combination with rhGH is expected to be more effective in reducing the symptoms of lipodystrophy. Further, it is contemplated that the statin may be effective in reducing the dosage and frequency of rhGH administration needed to produce the therapeutic effects.

Collective side effects of rhGH administration include swelling of the fingers or paresthesia due to tissue turgor, a few transient elevations of fasting glucose and triglycerides. It is contemplated that the use of the statin-based therapeutic agent may ameliorate these side effects of rhGH therapy. At 12 weeks, total cholesterol and fasting triglycerides dropped significantly, while HDL cholesterol and glucose increased, but none of these changes were deemed clinically significant (Engleson et al., Antiviral Therapy 4:(Sup 2):11 [Abstract 006], 1999; Engleson et al., Am J Clin Nutr 69(6):1162-1169, 1999). No additional episodes of hypertension or elevated pancreatic enzymes have been reported.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

What is claimed Is:

1. A method of treating a human suffering from an abnormal lipid distribution disorder, the method comprising administering to said subject a growth hormone and a statin-based therapeutic agent.

2. The method of claim 1, wherein said statin-based agent and said growth hormone are provided in a single pharmaceutical composition.

3. The method of claim 1, wherein said statin-based agent is provided in a first pharmaceutical composition and said growth hormone is provided in a second pharmaceutical composition.

4. The method of claim 1, wherein said growth hormone is recombinant growth hormone.

5. The method of claim 1, wherein said growth hormone has been isolated from an animal.

6. The method of claim 1, wherein said statin-based agent is a lovastatin or a lovastatin analog.

7. The method of claim 1, wherein said statin-based drug is selected from the group consisting of atorvastatin, pravastatin, simvastatin, lovastatin, and fluvastatin.

8. The method of claim 1, wherein said abnormal lipid distribution disorder is non-HIV-related lipodystrophy.

9. The method of claim 1, wherein said abnormal lipid distribution disorder is an HIV-related abnormal lipid distribution disorder.

10. The method of claim 9, wherein said HIV-related abnormal lipid distribution disorder is selected from the atherogenic dyslipidemia, hypertriglyceridemia, elevated levels of cholesterol, elevated levels of low-density-lipoprotein cholesterol, and low levels of high-density lipoprotein cholesterol.

11. The method of claim 1, wherein said subject manifests a symptom associated with diabetes related adiposity.

12. The method of claim 11, wherein said symptom is selected from the group consisting of insulin resistance, beta-cell dysfunction, loss of first phase insulin secretion, impaired glucose tolerance (IGT), elevated endogenous glucose production, excessive gluconeogenesis.

13. The method of claim 1, wherein said subject is suffering from Type 2 Diabetes.

14. The method of claim 11, wherein subject is further treated for diabetes, the method comprising administering an insulin secretagogue.

15. The method of claim 14, wherein said insulin secretagogue is selected from the group consisting of a sulphonylurea; tolbutamide; chlorpropamide; glimepiride; glipizide; glyburide; a meglitinides; repaglinide; pramlintide; morphilinoguanide; acetylcholine; a muscarinic agonist; carbachol; bethanechol; beta-L-glucose pentaacetate; chiro-inositol; myo-inositol; GIP; GLP-1; and Extendin-4.

16. The method of claim 15, wherein said insulin secretagogue is a non-glucose dependent insulin secretagogue, and the combined effect of administering said growth hormone, statin and insulin secretagogue produces insulin release patterns capable of attaining glucose dependent, bi-phasic release characteristics with reduced likelihood of producing hypoglycemia.

17. The method of claim 1, wherein said subject is further treated with leptin.

18. A therapeutic agent for use in combination therapy for an abnormal lipid distribution disorder, said composition comprising:
a. a first composition comprising a recombinant growth hormone in a pharmaceutically acceptable carrier, excipient or diluent; and
b. a second composition comprising a statin-based drug in a pharmaceutically acceptable carrier, excipient or diluent.

19. The therapeutic agent of claim 18, wherein said growth hormone and said statin-based drug are formulated in a single formulation.

20. The therapeutic agent of claim of claim 18, wherein said growth hormone is formulated in a separate formulation from said statin-based drug formulation.

21. The therapeutic agent of claim 20, wherein said growth hormone formulation and said statin-based drug formulation are formulated as injectable formulations.

22. The therapeutic agent of claim 20, wherein said statin-based drug formulation is formulated for oral administration.

23. The therapeutic agent of claim 20, wherein said statin-based drug is a lovastatin or an analog thereof.

24. The therapeutic agent of claim 20, wherein said statin-based drug is selected from the group consisting of atorvastatin, pravastatin, simvastatin, lovastatin, and fluvastatin.
